(19) United States
(12) Patent Application Publication
(10) Pub. No.: US 2015/0320680 A1
(43) Pub. Date: Nov. 12, 2015

(54) INJECTABLE COMPOSITION CONTAINING CHLOROTHIAZIDE

(71) Applicant: GETZ PHARMA RESEARCH PVT. LTD., Thane (IN)

(72) Inventors: Jeffrey Bauer, Thane (IN); Sivakumar Venkata Bobba, Thane (IN); Alok Pramod Tripathi, Thane (IN); Vinodkumar Gurunath Indure, Thane (IN)

(73) Assignee: GETZ PHARMA RESEARCH PVT. LTD., Thane (IN)

(21) Appl. No.: 14/648,963

(22) PCT Filed: Jan. 7, 2014
(86) PCT No.: PCT/IB14/00009

§ 371 Date: Jun. 2, 2015

(30) Foreign Application Priority Data
Jan. 9, 2013 (IN) 74/MUM/2013

(51) Int. Cl. A61K 9/00; A61K 31/549

(52) U.S. Cl. CPC A61K 9/0019; A61K 31/549

(57) ABSTRACT

The present invention relates to stable ready to use injectable liquid composition of chlorothiazide or its pharmaceutically acceptable salts.

### FIELD OF THE INVENTION

The field relates to ready to use stable injectable liquid composition of chlorothiazide or its pharmaceutically acceptable salts and methods of their preparation.

### BACKGROUND OF THE INVENTION

Chlorothiazide sodium is a diuretic and antihypertensive. Chemically chlorothiazide sodium is the monosodium salt of 6-chloro-2H-1,2,4-benxothiadiazine-7-sulfonamide 1,1-dioxide and its molecular weight is 317.71. Its empirical formula is C₇H₅ClN₃NaO₄S₂ and it is represented by compound of structural formula I.

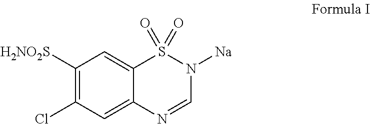

Chlorothiazide sodium injectable injection has been approved in USA prior to Jan. 1, 1982 and is indicated as adjunctive therapy in edema associated with congestive heart failure, hepatic cirrhosis, and corticosteroid and estrogen therapy and also useful in edema due to various forms of renal dysfunction such as nephrotic syndrome, acute glomerulonephritis, and chronic renal failure.

The proprietary name of chlorothiazide sodium injection is Diuril, which is approved to Oak Pharms, Akorn in USA. The marketed dosage form of chlorothiazide sodium for Injection is a sterile lyophilized white powder, which is supplied in vials and each vial containing chlorothiazide sodium equivalent to 0.5 gm of chlorothiazide base and inactive ingredients as mannitol and sodium hydroxide to adjust pH.

Chlorothiazide sodium for injection is given slowly by direct intravenous injection or by intravenous infusion wherein 18 ml of sterile water for injection is added to the vial to form an isotonic solution for intravenous injection. The reconstituted solution is compatible with dextrose or sodium chloride solutions for intravenous infusion and is being used immediately after reconstitution.

Chlorothiazide is also available in different dosage form such as oral suspension and oral tablet.

U.S. Pat. No. 4,713,238 discloses water soluble chlorothiazide in a complexed state which is derived from the reaction between N-Vinyl lactam polymer and chlorothiazide in alkaline media. The chlorothiazide in the complexed state exhibits at least a 50 fold increase in water solubility over the uncomplexed compound.

U.S. Patent Publication No. 20110263579 discloses generically parenteral dosage form of chlorothiazide sodium which may be in the form of solution or as a lyophilized product. However lyophilized product is exemplified.

U.S. Patent Publication No. 20120277249 (hereinafter referred to as U.S.'249discloses non-aqueous, homogeneous solution comprising a solubilized lipophilic pharmaceutical agent and an amphophilic liquid polymeric solvent, the formulation being essentially free of non-polymeric organic solvents, water and non-solubilized particles, wherein the solution remains stable and essentially free of non-solubilized particles. The composition is further diluted with a desired aqueous diluent such as an infusion fluid for parenteral administration to a subject such as a human. However, U.S.'249 cover chlorothiazide or its salts formulation generically and specific formulation of chlorothiazide or its pharmaceutically acceptable salt is not exemplified.

The PCT publication No. 2011016049 discloses pharmaceutical preparation comprising gemcitabine or its pharmaceutical acceptable salts in a ready-to-use form.

The PCT publication No. 2009133455 discloses pharmaceutical composition containing clopidogrel or its pharmaceutically acceptable salts in the form of ready to use solution.

The applicant of the present invention observed that chlorothiazide sodium sterile lyophilized white powder degrades after reconstitution with water.

The preparation of sterile chlorothiazide sodium lyophilized powder involves “Lyophilizer” which is expensive equipment at commercial scale and therefore the sterile chlorothiazide sodium lyophilized powder is an expensive dosage form.

Accordingly there is a need in the art to develop simple, stable, non-expensive dosage form of chlorothiazide or its pharmaceutically acceptable salts.

The present applicant invented ready to use stable injectable liquid composition of chlorothiazide or its pharmaceutically acceptable salts.

### SUMMARY OF THE INVENTION

A first aspect of the present invention is to provide ready to use stable injectable liquid composition of chlorothiazide or its pharmaceutically acceptable salts.

Another aspect of the present invention is to provide ready to use stable injectable liquid composition of chlorothiazide or its pharmaceutically acceptable salts along with one or more pharmaceutically acceptable excipients or mixture thereof.

Another aspect of the present invention is to provide ready to use stable non-aqueous injectable pharmaceutical composition comprising chlorothiazide or its pharmaceutically acceptable salts.

Another aspect of the present invention is to provide ready to use stable non-aqueous injectable pharmaceutical composition comprising chlorothiazide or its pharmaceutically acceptable salts along with one or more pharmaceutically acceptable excipients or mixture thereof.

Another aspect of the present invention is to provide process of preparing ready to use stable injectable liquid composition of chlorothiazide or its pharmaceutically acceptable salts.

Another aspect of the present invention is to provide process of preparing ready to use stable non-aqueous injectable pharmaceutical composition comprising chlorothiazide or its pharmaceutically acceptable salts.

Another aspect of the present invention is to provide ready to use stable non-aqueous injectable pharmaceutical composition comprising chlorothiazide or its pharmaceutically acceptable salts used as adjunctive therapy in edema associated with congestive heart failure, hepatic cirrhosis, and corticosteroid and estrogen therapy and also for the treating edema due to various forms of renal dysfunction such as nephrotic syndrome, acute glomerulonephritis, and chronic renal failure in human being.

### DETAILED DESCRIPTION OF THE INVENTION

The examples and drawings provided in the detailed description are merely examples, and should not be used to limit the scope of the claims in any claim construction or interpretation.

The “ready to use stable injectable liquid composition of chlorothiazide or its pharmaceutically acceptable salts” refers to use of stable injectable liquid composition obviating the need of reconstitution with sterile water.

The “ready to use stable injectable liquid composition of chlorothiazide or its pharmaceutically acceptable salts” is compatible with dextrose or sodium chloride solutions for intravenous infusion and is being used directly without reconstitution.

The “ready to use stable injectable liquid composition of chlorothiazide or its pharmaceutically acceptable salts” comprises non-aqueous solvents or mixture(s) thereof.

The non-aqueous solvent is selected from the group comprising of methanol, ethanol, polyethylene glycol, propylene glycol or mixture(s) thereof.

The chlorothiazide can be present in anhydrous form or in hydrate form.

The pharmaceutically acceptable salts of chlorothiazide includes but not limited to chlorothiazide sodium.

The chlorothiazide sodium can be present in anhydrous form or in hydrate form.

The chlorothiazide sodium can be used as such or it can be prepared in-situ by the reaction of chlorothiazide and sodium ethoxide.

The ready to use stable injectable liquid composition of chlorothiazide or its pharmaceutically acceptable salts can have a pH in the range of 2 to 10.

The ready to use stable injectable liquid composition of chlorothiazide or its pharmaceutically acceptable salts is present in the form of clear solution essentially free from visible particles.

The ready to use stable injectable liquid composition of chlorothiazide or its pharmaceutically acceptable salts may be administered by intravenous injection or by intravenous infusion.

The ready to use stable injectable liquid composition of chlorothiazide or its pharmaceutically acceptable salts may contain one or more excipients selected from the group consisting of suitable pH adjustifier, tonicity modifier, crystal growth inhibitor, buffering agent, solubilizing agent, preservative or antioxidants.

The pH adjustifier may be selected from the group comprising of sodium hydroxide, sodium carbonate, hydrochloric acid, lactic acid or mixture(s) thereof.

The tonicity modifier may be selected from the group comprising of sodium chloride, magnesium chloride, mannitol, dextrose or mixture(s) thereof.

The crystal growth inhibitor may be selected from the group comprising of polyvinylpyrrolidone, hydroxy propyl cellulose, polyethylene glycol, dimethyl sulfoxide or mixture(s) thereof.

The buffering agent may be selected from the group comprising of sodium succinate, gluconate, histidine, citrate phosphate, sodium citrate, citric acid or mixture(s) thereof.

The solubilizing agent may be selected from the group comprising of surfactants, cyclodextrin or mixture(s) thereof.

The surfactants may be selected from the group comprising of α-tocopherol, polyethylene glycol succinate, monomethoxy polyethylene glycolpolylactide, polyethylene glycol-15-hydroxystearate, polyoxyethylenesorbitan fatty acid ester, polyoxyethylene-polyoxypropylene copolymer, poloxamers, sodium lauryl sulphate or mixture(s) thereof.

The cyclodextrin may be selected from the group comprising of α-cyclodextrin, 2-hydroxypropyl-β-cylodedextrin, sulfobutylether-1-β-cyclodextrin, sulfobutyl ether-4-β-cyclodextrin, sulfobutyl ether-7-β-cylcodextrin or mixture(s) thereof.

The preservative may be selected from the group comprising of chlorocresol, benzyl alcohol, ethanol, bronopol, sucrose, chlorhexidine gluconate, thimerosal, benzethonium chloride, benzalkonium chloride, chlorobutanol, benzoic acid, meta-cresol, phenol or mixture(s) thereof.

The antioxidants may be selected from the group comprising of butylated hydroxyl toluene, bulylated hydroxyanisole, tocopherols such as alpha tocopherol, propyl gallate, ascorbates, ascorbic acid, sodium ascorbate, potassium or sodium salts of sulphurous acid or mixture(s) thereof.

The present invention provides the process of preparing stable injectable liquid composition of chlorothiazide or its pharmaceutically acceptable salts wherein process involve the steps comprising dissolving chlorothiazide or its pharmaceutically acceptable salts and one or more pharmaceutically acceptable excipient(s) in non-aqueous solvent. The sequence of mixing excipients or active ingredient in non-aqueous solvent may vary. Further one or more excipients optionally such as pH adjustifier, tonicity modifier, crystal growth inhibitor, buffering agent, solubilizing agent and antioxidants may be added and the resulting solution was filtered through suitable filter and filled into the vials or PFS (prefilled syringe).

Stability of the pharmaceutical composition of the present invention was tested at initial stage and after stability storage by subjecting the samples under various storage conditions such as: 40° C.±2° C./75% RH±5% RH, 25° C.±2° C./60% RH±5% RH and 2-8° C.

RH mentioned herein refers Relative Humidity.

HPLC method for analyzing samples of chlorothiazide or its pharmaceutically acceptable salts:

Analysis of the chlorothiazide or its pharmaceutically acceptable salts samples was performed using high pressure liquid chromatography (HPLC), the experimental parameters of which are given below.
Column: Phenomenex Luna Silica (2) 100Å, 250×4.6 mm, 5 μ
Mobile Phase A: n-Hexane
Mobile Phase B: Ethanol
Diluent: 100% Ethanol
Flow rate: 1.0 mL/min
Column temperature: 40° C.
Sample temperature: 10 ° C.

Injection Volume: 5 μL
Wavelength: 281 nm
Run time: 50 minutes
Retention Time: About 15.0 minutes
Gradient Program :

| Time in | Mobile phase | Mobile phase |
|---|---|---|
| Minutes | A (% v/v) | B (% v/v) |
| 0.0 | 80 | 20 |
| 25.0 | 60 | 40 |
| 40.0 | 30 | 70 |
| 42.0 | 80 | 20 |
| 50.0 | 80 | 20 |

A ready to use stable injectable liquid composition of chlorothiazide or its pharmaceutically acceptable salts of present invention is found stable at 40° C.±2° C./75% RH±5% RH, 25° C.±2° C./60% RH±5% RH and 2-8° C.

### EXAMPLES

In the following examples, the preferred embodiments of the present invention are described only by way of illustrating the process of the invention. However, these are not intended to limit the scope of the present invention in any way.

### Example 1

A. Formula of chlorothiazide anhydrous/salt Injection:

| Sr. | | | Quantity/ | | Quantity/ | |
|---|---|---|---|---|---|---|
| No. | Ingredients | % w/w | 18 mL | | batch (gm) | |
| 1 | Chlorothiazide Anhydrous | 2.802 | 504.4 | mg | 2.241 | gm* |
| 2 | Polyethylene glycol 300 | 55.55 | 9.99 | mL | 44.44 | mL |
| | (Croda) | | | | | |
| 3 | Butylated Hydroxytoluene | 0.002 | 0.36 | mg | 0.0016 | gm |
| 4 | Butylated Hydroxyanisole | 0.0002 | 0.036 | mg | 0.00016 | gm |
| 5 | Absolute Alcohol | q.s to | q.s to | | q.s to | |
| | | 100% | 18 | mL | 80 | mL |

*The quantity of [[c]]Chlorothiazide Anhydrous after assay and water content consideration.

B. Manufacturing Procedure:

1. Accurately weighed polyethylene glycol 300 was transferred to schott bottle.

2. Accurately weighed chlorothiazide anhydrous was transferred to schott bottle containing polyethylene glycol 300. Nitrogen was flushed into the schott bottle & cap was fitted tightly and stirred for 1 hour.

3. 13.33 mL of BHT-BHA solution was transferred from stock solution to step 2. Nitrogen was flushed into the schott bottle & cap was fitted tightly and stirred for 45 minutes. A clear solution was obtained.

4. Volume was made up to 80 mL using absolute alcohol. Nitrogen was flushed into the schott bottle & cap was fitted tightly and stirred for 10 minutes.

5. Filtration was performed using 0.22 μ PVDF filter.

6. Batch was loaded on stability in clear type I glass vial 10 mL & sealed with rubber stopper & aluminium cap.

Preparation of BHT (0.12 mg/mL) & BHA (0.012 mg/mL) Stock solution:

a. 120 mg of BHT & 12 mg of BHA was dissolved in small amount of absolute alcohol & volume was made up to 100 mL using absolute alcohol.

b. 10 mL from step (a) was taken & diluted to 100 mL using absolute alcohol to obtain stock solution of BHT (0.12 mg/mL) & BHA (0.012 mg/mL).

C. Stability Results:

| | | | 1 Month | | | 3 Month | | |
|---|---|---|---|---|---|---|---|---|
| Sr. | Tests/ | | | 25° C. ± 2° C./ | 40° C. ± 2° C./ | | 25° C. ± 2° C./ | 40° C. ± 2° C./ |
| No | Parameters | Initial | 2-8° C. | 60% RH ± 5% RH | 75% RH ± 5% RH | 2-8° C. | 60% RH ± 5% RH | 75% RH ± 5% RH |
| 1. | Description | Light | Light | Light | Light | Light | Light | Light |
| | | yellow | yellow | yellow | yellow | yellow | yellow | yellow |
| | | colored | colored | colored | colored | colored | colored | colored |
| | | viscous | viscous | viscous | viscous | viscous | viscous | viscous |
| | | liquid | liquid | liquid | liquid | liquid | liquid | liquid |
| 2. | Related Substances (%) | | | | | | | |
| a) | Disulphonamide | ND | 0.02 | 0.02 | 0.016 | 0.022 | 0.021 | 0.024 |
| b) | Single Max unknown | 0.018 | ND | 0.013 | 0.064 | 0.006 | 0.034 | 0.115 |
| c) | Total Impurities | 0.018 | 0.02 | 0.033 | 0.105 | 0.028 | 0.112 | 0.212 |

ND: Not Detected

### Example 2

A. Formula of chlorothiazide Anhydrous Injection:

| Sr. | | | Quantity/ | | Quantity/ | |
|---|---|---|---|---|---|---|
| No. | Ingredients | % w/w | 18 mL | | batch (gm) | |
| 1 | Chlorothiazide Anhydrous | 2.802 | 504.4 | mg | 7.005 | gm* |
| 2 | Polyethylene glycol 300 | 55.55 | 9.99 | mL | 138.88 | mL |
| | (NOF) | | | | | |
| 3 | Butylated Hydroxytoluene | 0.002 | 0.36 | mg | 0.005 | gm |
| 4 | Butylated Hydroxyanisole | 0.0002 | 0.036 | mg | 0.0005 | gm |
| 5 | Absolute Alcohol | q.s to | q.s to | | q.s to | |
| | | 100% | 18 | mL | 250 | mL |

*The quantity of chlorothiazide Anhydrous after assay and water content consideration.

B. Manufacturing Procedure:

1. Accurately weighed polyethylene glycol 300 was transferred to schott bottle.

2. Accurately weighed chlorothiazide anhydrous was transferred to schott bottle containing polyethylene glycol 300. Nitrogen was flushed into the schott bottle & cap was fitted tightly and stirred for 2 hour.

3. 41.67 mL of BHT-BHA solution was transferred from stock solution to step 2. Nitrogen was flushed into the schott bottle & cap was fitted tightly and stirred for 30 minutes. A clear solution was obtained.

4. Volume was made up to 250 mL using absolute alcohol. Nitrogen was flushed into the schott bottle & cap was fitted tightly and stirred for 10 minutes.

5. Filtration was performed using 0.22 μ PVDF filter.

6. Batch was loaded on stability in clear type I glass vial 10 mL & sealed with rubber stopper & aluminium cap.

Preparation of BHT (0.12 mg/mL & BHA (0.012 mg/mL) Stock solution:

a. 120 mg of BHT & 12 mg of BHA was dissolved in small amount of absolute alcohol & volume was made up to 100 mL using absolute alcohol.

b. 10 mL from step (a) was taken & diluted to 100 mL using absolute alcohol to obtain stock solution of BHT (0.12 mg/mL) & BHA (0.012 mg/mL).

C. Stability Results:

| | | | 1 Month | | | 3 Month | | |
|---|---|---|---|---|---|---|---|---|
| Sr. | Tests/ | | | 25° C. ± 2° C./ | 40° C. ± 2° C./ | | 25° C. ± 2° C./ | 40° C. ± 2° C./ |
| No | Parameters | Initial | 2-8° C. | 60% RH ± 5% RH | 75% RH ± 5% RH | 2-8° C. | 60% RH ± 5% RH | 75% RH ± 5% RH |
| 1. | Description | Light | Light | Light | Light | Light | Light | Light |
| | | yellow | yellow | yellow | yellow | yellow | yellow | yellow |
| | | colored | colored | colored | colored | colored | colored | colored |
| | | viscous | viscous | viscous | viscous | viscous | viscous | viscous |
| | | liquid | liquid | liquid | liquid | liquid | liquid | liquid |
| 2. | pH | 6.70 | 5.38 | 5.37 | 5.30 | 4.76 | 4.75 | 4.8 |
| 3. | Related Substances (%) | | | | | | | |
| a) | Disulphonamide | 0.019 | 0.018 | 0.019 | 0.019 | 0.02 | 0.022 | 0.024 |
| b) | Single Max unknown | ND | BDL | 0.013 | 0.064 | 0.006 | 0.40 | 0.132 |
| c) | Total Impurities | 0.019 | 0.018 | 0.032 | 0.083 | 0.026 | 0.062 | 0.164 |
| 4. | Water content | 1.6 | 2.0 | 2.0 | 2.0 | 1.8 | 1.2 | 1.8 |
| | (%) | | | | | | | |

ND: Not Detected
BDL: Below Detection Limit

### Example 3

A. Formula of chlorothiazide sodium dihydrate Injection:

| Sr. | | | Quantity/ | | Quantity/ | |
|---|---|---|---|---|---|---|
| No. | Ingredients | % w/w | 18 mL | | batch (gm) | |
| 1 | Chlorothiazide sodium | 3.35 | 603.9 | mg | 8.388 | gm* |
| | dihydrate | | | | | |
| 2 | Polyethylene Ltlycol 300 | 55.55 | 9.99 | mL | 138.88 | mL |
| | (NOF Corp) | | | | | |
| 3 | Butylated Hydroxytoluene | 0.002 | 0.36 | mg | 0.005 | gm |
| 4 | Butylated Hydroxyanisole | 0.0002 | 0.036 | mg | 0.0005 | gm |
| 5 | Absolute Alcohol | q.s to | q.s to | | q.s to | |
| | | 100% | 18 | mL | 250 | mL |

*The quantity of chlorothiazide Sodium Dihydrate alter assay and water content consideration.

B. Manufacturing process:

1. Accurately weighed polyethylene glycol 300 was transferred to schott bottle.

2. Accurately weighed chlorothiazide sodium dihydrate was transferred to schott bottle containing polyethylene glycol 300. Nitrogen was flushed into the schott bottle & cap was fitted tightly and stirred for 2 hour.

3. 41.67 mL of BHT-BHA solution was transferred from stock solution to step 2. Nitrogen was flushed into the schott bottle & cap was fitted tightly and stirred for 30 minutes. A clear solution was obtained.

4. Volume was made up to 250 mL using absolute alcohol. Nitrogen was flushed into the schott bottle & cap was fitted tightly and stirred for 10 minutes.

5. Filtration was performed using 0.22 μ PVDF filter.

6. Batch was loaded on stability in clear type I glass vial 10 mL & sealed with rubber stopper & aluminium cap.

Preparation of BHT (0.12 mg/mL) & BHA (0.012 mg/mL) Stock solution:

a. 120 mg of BHT & 12 mg of BHA was dissolved in small amount of absolute alcohol & volume was made up to 100 mL using absolute alcohol.

b. 10 mL from step (a) was taken & diluted to 100 mL using absolute alcohol to obtain stock solution of BHT (0.12 mg/mL) & BHA (0.012 mg/mL).

C. Stability Results:

| | | | 1 Month | | | 3 Month | | |
|---|---|---|---|---|---|---|---|---|
| Sr. | Tests/ | | | 25° C. ± 2° C./ | 40° C. ± 2° C./ | | 25° C. ± 2° C./ | 40° C. ± 2° C./ |
| No | Parameters | Initial | 2-8° C. | 60% RH ± 5% RH | 75% RH ± 5% RH | 2-8° C. | 60% RH ± 5% RH | 75% RH ± 5% RH |
| 1. | Description | Light | Light | Light | Light | Light | Light | Light |
| | | yellow | yellow | yellow | yellow | yellow | yellow | yellow |
| | | colored | colored | colored | colored | colored | colored | colored |
| | | viscous | viscous | viscous | viscous | viscous | viscous | viscous |
| | | liquid | liquid | liquid | liquid | liquid | liquid | liquid |
| 2. | pH | 7.3 | 8.97 | 9.14 | 9.15 | 8.34 | 8.96 | 9.17 |
| 3. | Related Substances (%) | | | | | | | |
| a) | Disulphonamide | ND | BDL | BDL | 0.071 | ND | 0.018 | 0.247 |
| b) | Single Max unknown | ND | 0.041 | 0.173 | 0.643 | 0.105 | 0.501 | 0.935 |
| c) | Total Impurities | 0.0 | 0.041 | 0.173 | 0.714 | 0.105 | 0.519 | 1.233 |
| 4. | Water content | 1.9 | 2.4 | 2.2 | 2.0 | 2.2 | 2.2 | 2.1 |
| | (%) | | | | | | | |

ND: Not Detected
BDL: Below Detection Limit

### Example 4

A. Formula of Chlorothiazide Sodium Injection Using Sodium Ethoxide

| Sr. | | | Quantity/ | | Quantity/ | |
|---|---|---|---|---|---|---|
| No. | Ingredients | % w/w | 18 mL | | batch (gm) | |
| 1 | Chlorothiazide Anhydrous | 2.802 | 504.4 | mg | 2.802 | gm* |
| 2 | Polyethylene glycol 300 | 55.55 | 9.9 | mL | 55.55 | mL |
| 3 | Butylated Hydroxytoluene | 0.002 | 0.36 | mg | 0.002 | gm |
| | (BHT) | | | | | |
| 4 | Butylated Hydroxyanisole | 0.0002 | 0.036 | mg | 0.0002 | gm |
| | (BHA) | | | | | |
| 5 | Sodium Ethoxide^{#} | 0.670 | 120.74 | mg | 0.670 | gm |
| 6 | Absolute Alcohol | q.s to | q.s to | | q.s to | |
| | | 100% | 18 | mL | 100 | mL |

*The quantity of chlorothiazide Anhydrous after assay and water content consideration.
^{#}The quantity of Sodium Ethoxide after assay consideration.

B. Manufacturing procedure:

1. Accurately weighed polyethylene glycol 300 was transferred to schott bottle.

2. Accurately weighed chlorothiazide anhydrous was transferred to schott bottle containing polyethylene glycol 300. Nitrogen was flushed into the schott bottle & cap was fitted tightly and stirred for 2 hour.

3. Accurately weighed Sodium ethoxide was added in Alcohol (15% batch quantity) and dissolved and stirred for 10 min.

4. Add step 3 mixture to step 2 solution with nitrogen flushing and stirred for 1 hour.

5. BHT-BHA solution was transferred from stock solution to step 2. Nitrogen was flushed into the schott bottle & cap was fitted tightly and stirred for 30 minutes. A clear solution was obtained.

6. Volume was made up to 100 mL using absolute alcohol. Nitrogen was flushed into the schott bottle & cap was fitted tightly and stirred for 30 minutes.

7. Filtration was performed using 0.22 μ PVDF filter.

8. Batch was loaded on stability in clear type I glass vial 10 mL & sealed with rubber stopper & aluminium cap.

Preparation of BHT (0.12 mg/mL) & BHA (0.012 mg/mL) Stock solution:

a. 120 mg of BHT & 12 mg of BHA was dissolved in small amount of absolute alcohol & volume was made up to 100 mL using absolute alcohol.

b. 10 mL from step (a) was taken & diluted to 500 mL using absolute alcohol to obtain stock solution of BHT (0.12 mg/mL) & BHA (0.012 mg/mL).

C. Stability Results:

| | | | 2 Weeks | | | 1 Month | | |
|---|---|---|---|---|---|---|---|---|
| Sr. | Tests/ | | | 25° C. ± 2° C./ | 40° C. ± 2° C./ | | 25° C. ± 2° C./ | 40° C. ± 2° C./ |
| No | Parameters | Initial | 2-8° C. | 60% RH ± 5% RH | 75% RH ± 5% RH | 2-8° C. | 60% RH ± 5% RH | 75% RH ± 5% RH |
| 1. | Description | Light | Light | Light | Light | Light | Light | Light |
| | | yellow | yellow | yellow | yellow | yellow | yellow | yellow |
| | | colored | colored | colored | colored | colored | colored | colored |
| | | viscous | viscous | viscous | viscous | viscous | viscous | viscous |
| | | liquid | liquid | liquid | liquid | liquid | liquid | liquid |
| 2. | Related Substances (%) | | | | | | | |
| a) | Disulphonamide | 0.021 | 0.011 | 0.015 | 0.018 | 0.013 | 0.012 | 0.021 |
| b) | Single Max unknown | 0.006 | 0.026 | 0.032 | 0.090 | 0.033 | 0.035 | 0.121 |
| c) | Total Impurities | 0.027 | 0.037 | 0.079 | 0.167 | 0.046 | 0.087 | 0214 |

### Example 5

A. Formula of chlorothiazide Sodium Injection.

| Sr. | | | Quantity/ | | Quantity/ | |
|---|---|---|---|---|---|---|
| no. | Ingredients | % w/w | 18 mL | | batch (gm) | |
| 1 | Chlorothiazide sodium | 2.802 | 504.4 | mg | 2.802 | gm* |
| 2 | Polyethylene glycol 300 | 55.55 | 9.9 | mL | 55.55 | mL |
| 3 | Butylated Hydroxytoluene | 0.002 | 0.36 | mg | 0.002 | gm |
| 4 | Butylated Hydroxyanisole | 0.0002 | 0.036 | mg | 0.0002 | gm |
| 5 | Absolute Alcohol | q.s to | q.s to | | q.s to | |
| | | 100% | 18 | mL | 100 | mL |

*The quantity of chlorothiazide sodium after assay and water content consideration.

B. Manufacturing procedure:

1. Accurately weighed polyethylene glycol 300 was transferred to schott bottle.

2. Accurately weighed chlorothiazide sodium was transferred to schott bottle containing polyethylene glycol 300. Nitrogen was flushed into the schott bottle & cap was fitted tightly and stirred for 2 hour.

3. BHT-BHA solution was transferred from stock solution to step 2. Nitrogen was flushed into the schott bottle & cap was fitted tightly and stirred for 30 minutes. A clear solution was obtained.

4. Volume was made up to 100 mL using absolute alcohol. Nitrogen was flushed into the schott bottle & cap was fitted tightly and stirred for 30 minutes.

5. Filtration was performed using 0.22 μ PVDF filter.

6. Batch was loaded on stability in clear type I glass vial 10 mL & sealed with rubber stopper & aluminium cap.

Preparation of BHT (0.12 mg/mL) & BHA (0.012 mg/mL) Stock solution:

a. 120 mg of BHT & 12 mg of BHA was dissolved in small amount of absolute alcohol & volume was made up to 100 mL using absolute alcohol.

b. 10 mL from step (a) was taken & diluted to 100 mL using absolute alcohol to give stock solution of BHT (0.12 mg/mL) & BHA (0.012 mg/mL).

C. Stability Results:

| | | | 2 Weeks | | | 1 Month | | |
|---|---|---|---|---|---|---|---|---|
| Sr. | Tests/ | | | 25° C. ± 2° C./ | 40° C. ± 2° C./ | | 25° C. ± 2° C./ | 40° C. ± 2° C./ |
| No | Parameters | Initial | 2-8° C. | 60% RH ± 5% RH | 75% RH ± 5% RH | 2-8° C. | 60% RH ± 5% RH | 75% RH ± 5% RH |
| 1. | Description | Light | Light | Light | Light | Light | Light | Light |
| | | yellow | yellow | yellow | yellow | yellow | yellow | yellow |
| | | colored | colored | colored | colored | colored | colored | colored |
| | | viscous | viscous | viscous | viscous | viscous | viscous | viscous |
| | | liquid | liquid | liquid | liquid | liquid | liquid | liquid |
| 2. | Related Substances (%) | | | | | | | |
| a) | Disulphonamide | 0.015 | 0.011 | 0.011 | 0.012 | 0.010 | 0.007 | 0.017 |
| b) | Single Max unknown | 0.044 | 0.030 | 0.065 | 0.159 | 0.031 | 0.064 | 0.234 |
| c) | Total Impurities | 0.059 | 0.054 | 0.109 | 0.223 | 0.059 | 0.122 | 0.322 |

ND: Not Detected

The scope of the claims should not be limited by the preferred embodiments and examples, but should be given the broadest interpretation consistent with the specification as a whole.

1. (canceled)

2. (canceled)

3. A stable ready to use non-aqueous injectable pharmaceutical composition of chlorothiazide or its pharmaceutically acceptable salts consisting essentially of:
a) chlorothiazide or its pharmaceutically acceptable salts;
b) a non-aqueous solvent; and
c) optionally, a pH adjusting agent to adjust the pH between 2 to 10.

4. The stable ready to use injectable composition according to claim 3, wherein the composition is prepared by a process comprising the steps of:
a) mixing chlorothiazide or its pharmaceutically acceptable salts and one or more excipients in non-aqueous solvent;
b) filtering the resulting solution through suitable filter; and
c) filling the resulting filtered solution in a vial or prefilled syringe.

5. The stable ready to use injectable composition according to claim 3, wherein the pharmaceutically acceptable salt of chlorothiazide is chlorothiazide sodium.

6. The stable ready to use injectable composition according to claim 3, wherein the non-aqueous solvent is selected from the group consisting of methanol, ethanol, polyethylene glycol, propylene glycol and mixture(s) thereof.

7. The stable ready to use injectable composition according to claim 3 further comprising one or more pharmaceutically acceptable excipients.

8. The stable ready to use injectable composition according to claim 7, wherein the pharmaceutically acceptable excipients are selected from the group consisting of pH adjusting agent, tonicity modifier, crystal growth inhibitor, buffering agent, solubilizing agent, preservative, antioxidants and mixture(s) thereof.

9. The stable ready to use injectable composition according to claim 8, wherein the pH adjusting agent is selected from the group consisting of sodium hydroxide, sodium carbonate, hydrochloric acid, lactic acid and mixture(s) thereof.

10. The stable ready to use injectable composition according to claim 8, wherein the tonicity modifier is selected from the group consisting of sodium chloride, magnesium chloride, mannitol, dextrose and mixture(s) thereof.

11. The stable ready to use injectable composition according to claim 8, wherein the crystal growth inhibitor is selected from the group consisting of polyvinylpyrrolidone, hydroxy propyl cellulose, polyethylene glycol, dimethyl sulfoxide and mixture(s) thereof.

12. The stable ready to use injectable composition according to claim 8, wherein the buffering agent is selected from the group consisting of sodium succinate, gluconate, histidine, citrate phosphate, sodium citrate, citric acid and mixture(s) thereof.

13. The stable ready to use injectable composition according to claim 8, wherein the solubilizing agent is selected from the group consisting of surfactants, cyclodextrin and mixture(s) thereof.

14. The stable ready to use injectable composition according to claim 13, wherein the surfactant is selected from the group consisting of α-tocopherol, polyethylene glycol succinate, monomethoxy polyethylene glycolpolyactide, polyethylene glycol-15-hydroxystearate, polyoxyethylenesorbitan fatty acid ester, polyoxyethylene-polyoxypropylene copolymer, poloxamers, sodium lauryl sulphate and mixture(s) thereof.

15. The stable ready to use injectable composition according to claim 13, wherein the cyclodextrin is selected from the group consisting of α-cydodextrin, 2-hydroxypropyl-β-cyclodextrin, sulfobutylether-1-β-cyclodextrin, sulfobutyl ether-4-β-cyclodextrin, sulfobutyl ether-7-β-cyclodextrin and mixture(s) thereof.

16. The stable ready to use injectable composition according to claim 8, wherein the preservative is selected from the group consisting of chlorocresol, benzyl alcohol, ethanol, bronopol, sucrose, chlorhexidine gluconate, thimerosal, benzethonium chloride, benzalkonium chloride, chlorobutanol, benzoic acid, meta-cresol, phenol and mixture(s) thereof.

17. The stable ready to use injectable composition according to claim 8, wherein the antioxidants are selected from the group consisting of butylated hydroxyl toluene, butylated hydroxyanisole, tocopherols such as alpha tocopherol, propyl gallate, ascorbates, ascorbic acid, sodium ascorbate, potassium or sodium salts of sulphurous acid and mixture(s) thereof.

18. A ready to use stable non-aqueous injectable pharmaceutical composition according to claim 3, wherein the composition is for use as adjunctive therapy in edema associated with congestive heart failure, hepatic cirrhosis, and corticosteroid and estrogen therapy and also for the treating edema due to various forms of renal dysfunction such as nephrotic syndrome, acute glomerulonephritis, and chronic renal failure in human being.

19. A method for treating edema as an adjunctive therapy comprising: administering the stable ready to use non-aqueous injectable pharmaceutical composition according to claim 3.
